(19) United States
(12) Patent Application Publication
(10) Pub. No.: US 2017/0100191 A1
(43) Pub. Date: Apr. 13, 2017

(54) ELECTROCATHETER WITH ELECTRODES AT LEAST PARTIALLY MADE FROM PLASTIC MATERIAL

(71) Applicant: FIAB S.P.A., Vicchio (FI) (IT)

(72) Inventors: Alberto CALABRO', Firenze (IT); Antonio FASANO, Firenze (IT)

(21) Appl. No.: 15/290,614

(22) Filed: Oct. 11, 2016

(30) Foreign Application Priority Data
Oct. 12, 2015 (IT) 102015000060560

(51) Int. Cl. A61B 18/14; A61M 25/00

(52) U.S. Cl. CPC A61B 18/1492; A61M 25/0009; A61B 2018/1467; A61B 2018/00136

(57) ABSTRACT

The present invention relates to a electrocatheter for surgical operations with electrodes. It allows you to adjust the physical properties of the electrodes during the electrocatheter production, thus eliminating undesirable effects due to energy absorption by these electrodes, and which allows to obtain a remarkable reduction of production time of the electrocatheter.

The present invention relates to a electrocatheter for surgical operations with electrodes, which allows to adjust the physical properties of the electrodes during the electrocatheter production, thus eliminating undesirable effects due to absorption of electromagnetic energy by these electrodes, and which allows to obtain a considerable reduction of production electrocatheter times the same.

It is known that in the treatment of certain disorders, such as atrial fibrillation in particular, use is made of techniques, known as ablation, which intervene preferably with radiofrequency irradiation in the vicinity of the outlet of the pulmonary veins in the left atrium and other cardiac regions, for the purpose to restore the correct electrical behavior of the affected tissues and prevent the emergence of the phenomenon of fibrillation.

In these techniques, is therefore made use of a radio-frequency electromagnetic field emitted by a scaler electrode, which in addition to performing the task now said, produces an heating of tissue and of the surrounding parts, which should be limited and kept under control.

The propagation of heat in the surrounding tissue can cause such lesions in various organs (phrenic nerve, the vagus nerve, the atrial wall, esophagus). Particularly dangerous those governing the esophagus (known as atrio-esophageal fistula is an example of an almost always fatal injury). The temperature control in the esophageal lumen is possible via the insertion of a catheter with electrodes arranged along the catheter that are associated with thermal detectors.

These detectors are or comprise sensors that can be for example of the type with thermocouple or thermistor.

For each electrode may be present then also one or more detection conductors. Each of these conductors then comprises at least a heat-sensitive element of a respective sensor, which is associated to a respective electrode. Such heat-sensitive element is sensitive to the electrode temperature and/or the tissue in contact with or in proximity of the electrode itself. Each of these conductors is connected to the electrode by a part and the other part to the acquisition system for temperature detection.

The main body of the electrodes intended for heat detection is currently made of metal. Such metal can interfere with the electromagnetic field emitted by the ablation generator (the so-called “antenna” effect) giving rise to an undesired heating of the element itself with possible injuries induced in tissues in contact.

The object of the present invention is to develop a electrocatheter for surgical operations, which allows to select, during the electrocatheter production phase of the same, the material of the electrodes, in order to overcome the above concerns.

Additionally, the conductors that are connected to the electrodes are currently welded to the electrode material.

This determines the use of the welding material and the risk of damage or contaminate the electrodes and/or conductors.

Furthermore, the welding operation constitutes a further step of production, which determines an increase of the time required for the electrocatheter production.

Another object of the present invention is to develop a electrocatheter for surgical operations that will decrease the production time compared to currently known electrocatheters.

A further object of the present invention is to develop a electrocatheter for surgical operations that will decrease the risk of damage or contamination of the electrodes and/or conductors in the phase of electrocatheter production.

Such purposes are obtained by a electrocatheter for surgical operations, comprising at least one catheter, at least one electrode and at least a conductor connected to said at least one electrode, said electrode being arranged on said catheter, characterized in that said at least one electrode is made of at least partially with at least one plastic material.

In this way you can select that at least one plastic material to adjust the electrical conductivity and/or the thermal conductivity of the electrode in an optimized way for the operations to be performed by the electrocatheter itself.

The electrocatheter preferably comprises at least one sensor for detecting the temperature of said at least one electrode and/or of a human's body tissue.

Said sensor may comprise for example a thermistor or a thermocouple.

Such at least one conductor comprises at least a heat-sensitive element of said sensor.

Such at least one thermosensitive element is preferably integrated and/or incorporated in the electrode, at the time of formation of the latter.

In the case in which such a sensor is of the type with thermocouple, the electrocatheter comprises at least a further conductor in contact with said conductor and connected to said at least one electrode.

Such at least one further conductor comprises at least one additional heatsensitive element of said sensor.

Such at least one further heat-sensitive element is preferably integrated and/or incorporated in the electrode, at the time of formation of the latter.

Preferably the electrocatheter comprises a plurality of electrodes.

Preferably each of these electrodes is connected to a respective conductor and possibly to a respective additional conductor.

Each of these conductors may be suitable to transfer signals concerning of differences of electrical potentials or low-intensity current.

According to another aspect, the present invention relates to a method of producing a electrocatheter for surgical operations, comprising a step of providing at least one electrode and at least one conductor, in which during this phase said at least one electrode is made at least partially with at least one material plastic and at least a part of said at least one conductor is incorporated directly into the electrode body during the formation of said electrode.

Such a conductor that is incorporated in the electrode during the formation of the same, can be a conductor associated to a sensor or a conductor suitable for carrying current.

In case you want to create a sensor of the thermocouple type, during said step is arranged at least one further conductor which is also connected to said electrode and of which at least part is incorporated directly into the electrode body during said formation, so that said conductors are integrated and/or embedded in the electrode and in contact with each other.

Preferably said at least one plastic material is selected on the basis of the electrical and/or thermal conductivity desired electrode itself.

By adjusting the thermal conductivity at a high level, the invention can be used for instance in temperature measurements of various districts of living organisms without fear of altering the thermal inertia or interfere in temperature measurements during application of radio frequency energy.

More generally, by adjusting the electrical conductivity at low levels, can be minimized the effects of magnetic fields, also variable, allowing the use of electrodes in security for example in NMR applications (Nuclear Magnetic Resonance) and/or RF (Radio Frequency).

The features of the present invention will be clarified in the following detailed description, offered by way of example and not limitative of the most general concepts claimed.

The following detailed description refers to the attached drawing, in which:

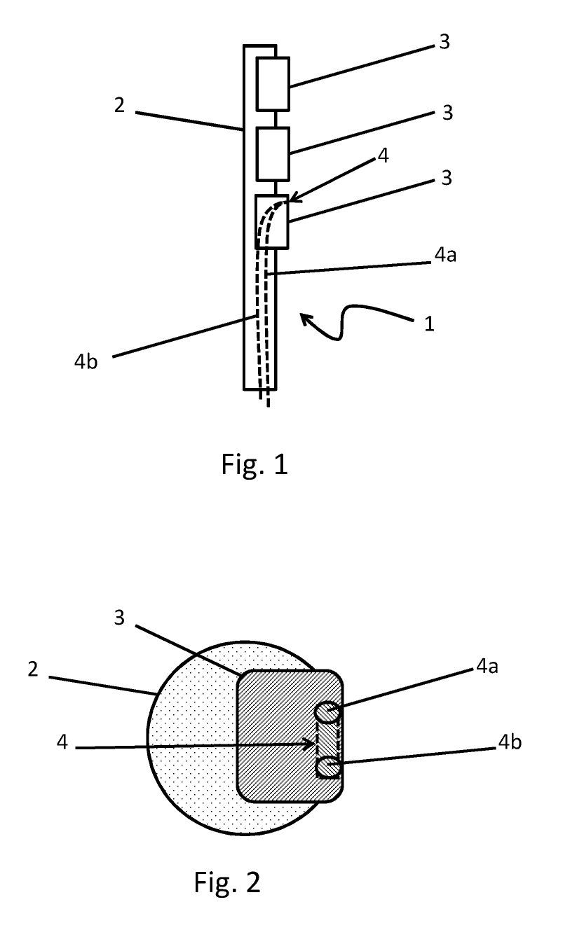

In FIG. 1 it is shown a schematization of a part of a electrocatheter according to a particular embodiment of the present invention, with some components removed for clarity;

In FIG. 2 is shown a electrocatheter section of FIG. 1.

In FIG. 1 it is shown a electrocatheter 1 for surgical operations, according to a particular embodiment of the present invention. Such electrocatheter 1 advantageously comprises at least an elongated body 2, preferably cylindrical, made of biocompatible material, such as silicone or polyurethane.

Such elongated body 2 can be referred for simplicity catheter.

The electrocatheter 1 comprises, in the embodiment shown, a plurality of electrodes 3, and at least one conductor 4a, which can be defined first conductor 4a. The first conductor 4a is connected to said at least one electrode 3.

In the embodiment shown, the electrocatheter 1 comprises at least a sensor 4 for detecting the temperature of at least one of said electrodes 3 and/or of a human body's tissue. Said first conductor 4a, in the embodiment shown, performs the function of sensing element of the sensor 4, and thus comprises at least a heat-sensitive element which can be defined first heat-sensitive element.

At least a portion of said first conductor 4a is integrated and/or embedded directly in the electrode 3.

Advantageously, this part of the first conductor 4a corresponds to this first heat-sensitive element, which is then integrated and/or incorporated directly in the electrode 3.

In another possible embodiment, the first conductor 4a may be used as a current transfer to the respective electrode 3.

In the embodiment shown, the sensor 4 may comprise for example a thermistor or a thermocouple.

In the case that sensor 4 is a thermocouple, the electrocatheter 1 comprises at least one further conductor 4b, which can be defined second conductor 4b.

This second conductor 4b, in case the sensor is a thermocouple 5, is in contact with the first conductor 4a, preferably in correspondence of a junction.

The second conductor 4b, in case the sensor is a thermocouple, is also connected to said at least one electrode 3.

The second conductor 4b, in case the sensor is a thermocouple, comprises at least one further element of said heat-sensitive sensor 4, which can be defined according heat-sensitive element.

At least a part of the second conductor 4b is integrated and/or embedded directly in the electrode 3.

Advantageously, this part of the second conductor 4b corresponds to this second thermosensitive element, which is then integrated and/or incorporated directly in the electrode 3.

Note that, in FIG. 1, the electrocatheter part 1 which is shown greatly simplified and schematic. The catheter 2 is shown only in part, and are only partially shown the conductors 4a and 4b connected to one of the electrodes 3.

FIG. 2 shows a electrocatheter 1 section transverse to the axis of development of the catheter 2, and in correspondence of a cross section of one of the electrodes 3. The electrodes 3 are preferably distributed along the development axis of the catheter 2.

FIG. 2 shows two cross sections of the respective conductors 4a and 4b.

It is noted that these first conductor 4a and 4b second conductor are at least partially embedded in the electrode 3, and in contact with each other along the dotted portion that connects these sections.

These conductors 4a and 4b may be, for example, electric wires or electric cables. For example, in case the sensor 4 is a thermocouple, the conductors 4a and 4b could advantageously be made of copper and constantan.

The dashed portion of FIG. 2 showing the sensor portion 4 that realizes the junction between the two conductors 4a and 4b.

Said portion is dashed because the cross section is considered a portion where the conductors 4a and 4b are not in contact, but some are already embedded in the electrode 3.

The sensor 4 may however be of another type than those described.

According to another aspect, the present invention concerns a method of production of a electrocatheter 1 for surgical operations, comprising a step of providing at least one electrode 3 and at least a conductor 4a connected to said electrode 3. During this phase, said at least one electrode 3 is made at least partially with at least one plastic material.

During this phase, at least a part of said at least one conductor 4a is incorporated directly into the electrode body 3 during the formation of said electrode 3.

Said at least a part of conductor 4a is preferably an its terminal part.

Depending on the chosen shape for the electrode, its production can be made by molding, forming, injection or another suitable technique to create objects made in at least one plastic material.

To obtain the electrocatheter 1 referred to the attached figures, during said step is arranged at least one further conductor 4b. Even this additional conductor 4b is advantageously connected to the electrode 3.

In particular, at least a part of the further conductor 4b is incorporated directly into the electrode body 3 during electrode formation itself, so that said conductors 4a and 4b are integrated and/or embedded in the electrode 3 and in contact with each other.

Said at least one part of the further conductor 4b is preferably an its terminal part.

Said at least one of plastic material of electrode 3 is selected on the basis of the electrical and\or thermal conductivity.

Preferably, in a time subsequent to this stage, the electrode 3 together with that part of the conductor 4a and/or to that part of the further conductor 4b, it is positioned on the catheter 2 so as to remain fixed at the same catheter 2.

More precisely, by way of example only, some of particular interest configurations may also be the following.

Another possible embodiment provides that the electrode includes a body and a coating located on at least part of the outer surface of said body.

This body is made by at least one metal material, and preferably such body is completely metallic.

Such a coating is realized by at least one plastic material, and preferably is fully realized by said at least one plastic material.

Such a coating is advantageously biocompatible and has very low electrical conductivity, but of sufficiently high thermal conductivity. The combination of these features ensures protection of the metallic body by the electromagnetic field without affecting the speed of response of the device to thermal signals.

The application of the coating can take place by immersion, coating, spray, electrostatic deposition, etc.

To obtain the latter possible embodiment, said body is prepared and is subsequently proceeds to the application or formation of such a coating by painting, spray, electrostatic deposition, or the like.

In a different embodiment, alternative to the application of the coating by paint, spray or similar, is possible to proceed to an appropriate chemical or electrochemical treatment to the outer surface of the electrode body.

In this case the formation of the coating takes place by chemical or electrochemical treatment on at least part of the outer surface of the electrode body.

Such treatment is advantageously performed so as to produce an electromagnetic shielding with very low thickness, so as not to degrade the thermal transmission property. An example of such treatment can be the galvanic oxidation, as with the anodized aluminum.

Such chemical or electrochemical treatment of the external surface of the body is suited to creating an electrically insulating layer so thin as not to compromise the speed of electrode response to temperature changes (as for example in aluminum anodized).

The electrode can also be composite, and comprises a light metal element, which supports the welding to the thermal sensor, and which is immersed or mounted on a main body in this case made of plastic material and with the features already specified.

It is noted that the electrode, as mentioned previously, can also be wholly realized by means of said at least one plastic material, preferably with the characteristics just described.

The invention attains the intended purposes, and allows to provide a product comprising a electrocatheter that eliminates the undesired interference between electrodes and/or conductors. This product ensures the preservation of the electrodes and the conductor end.

Furthermore they can also achieve the electrocatheter, according to one or more possible embodiments of the present invention, without welding operations.

In addition they also eliminate any residues that could contaminate the product.

1. Electrocatheter (1) for surgical operations, comprising at least one catheter (2), at least one electrode (3) and at least one conductor (4a) connected to said at least one electrode (3), said electrode (3) being disposed on said catheter (2), characterized in that said at least one electrode (3) comprises a metal body with at least one coating made from plastic material.

2. Electrocatheter according to claim 1, wherein at least a part of said at least one conductor is built into and/or incorporated in said at least one electrode.

3. Electrocatheter according to claim 1, comprising at least one sensor (4) for the detection of the temperature of said at least one electrode and/or of a tissue of a human body, said at least one conductor (4a) comprising at least one heat-sensitive element of said sensor.

4. Electrocatheter according to claim 3, wherein said sensor is a thermistor.

5. Electrocatheter according to claim 3, comprising at least one further conductor (4b) in contact with said conductor (4a) and connected to said at least one electrode (3), said at least one further conductor (4b) comprising at least one additional heat-sensitive element of said sensor (4), and
wherein at least a part of said at least one additional conductor is built into and/or incorporated in said at least one electrode.

6. Electrocatheter according to claim 5, wherein said sensor is a thermocouple.

7. Electrocatheter according to claim 1, wherein said at least one conductor is designed to transfer current to said at least one electrode.

8. Electrocatheter according to claim 1, comprising a plurality of electrodes.

9. A method for the production of a electrocatheter for surgical operations, comprising a step of providing at least one electrode and at least a conductor connected to said electrode, wherein during this step said at least one electrode is made at least partially from one metal and is subsequently proceeds to the formation or application of at least a coating, by a chemical or electrochemical treatment, on at least part of the outer surface of said body, said coating being made of said at least one plastic material.

10. Method according to claim 9, wherein at least a part of said at least one conductor is incorporated directly into the electrode body during the formation of said electrode.

11. Method according to claim 9, wherein during said step is arranged at least one additional conductor connected to said electrode and of which at least part is incorporated directly into the electrode body during the formation, so that said conductors are at least partially built into and/or incorporated in the electrode and in contact with each other.

12. Method according to claim 9, wherein said formation or application is performed by painting, spray, electrostatic deposition or the like, so as to produce an electromagnetic shielding with very low thickness, so as not to degrade the thermal transmission property.

13. Method according to claim 9, wherein said at least one plastic material is selected on the basis of the electric and/or desired thermal conductivity, said electrical conductivity being preferably very low and said thermal conductivity being preferably high.
